(19) United States
(12) Patent Application Publication
(10) Pub. No.: US 2013/0064897 A1
(43) Pub. Date: Mar. 14, 2013

(54) New process for producing hydrodispexible dry pharmaceutical composition and the pharmaceutical compositions thus obtained

(71) Applicant: Patrice Binay, Couchey (FR)

(72) Inventor: Patrice Binay, Couchey (FR)

(21) Appl. No.: 12/452,543

(22) PCT Filed: Jul. 7, 2008
(86) PCT No.: PCT/FR2008/000973

§ 371 Date: Jan. 19, 2010

(30) Foreign Application Priority Data
Jul. 6, 2007 (FR) 0704874

(51) Int. Cl. A61K 9/16; B05D 5/00; A61K 31/343; A61K 31/485; A61K 31/216; A61K 31/57

(52) U.S. Cl. 424/498; 514/543; 514/177; 514/469; 514/279; 427/2.15

(57) ABSTRACT

The invention relates to life's necessities and in particular those relating to health, more particularly a method for dissolving and improving the intestinal absorption of active ingredients which are poorly-water-soluble or water-insoluble or which cannot be turned into salts in gastric juice, containing one or more active ingredients dispersed in a polyoxyethylene 32 fatty acid ester then hot spraying said dispersion onto a granular excipient in a fluid bed. The powder mixture thus formed is distributed in pharmaceutical compositions after optional dilution in a pharmaceutically-acceptable non-toxic inert excipient. The above is of use in the production of pharmaceutical compositions containing one or more pharmaceutically-acceptable non-toxic inert excipients.

It has been known many years ago, that through the side of classical explorations of the pharmacological and toxicological properties of the drugs, one of the determining factors of the activity is the quantitative aspect of the resorption of the active ingredient. The kinetics becomes more and more the subject of systematic studies, due to the fact that the intensity of the answer and frequently its nature are dependant on the concentration reached at the level of the receptor organ. The significance of these studies on the pharmacokinetics and consequently on the bioavailability, has shown the full interest of the modifications, namely of galenical manner performed, in the course of the production in view of obtaining those the most suitable for the administration through digestive way.

The major problem of the weak intestinal resorption of the drugs seems to be linked to the fact that that takes place during a short period of time in a reduced area of the intestines. To increase the resorption, it is of significance that the active ingredient will be conveyed therein shortly and in a form made soluble.

The drugs which haves a limited solubility in water or are poorly salifiable during this passage in the stomach, so as they are only partially resorbed. The previous literature indicated that the resorption through digestive way may be altered in a favourable manner by the study of the size of particles by the addition namely of non-ionic tensioactive agents, as well as by the addition of a solubilizing agent.

On the other side the micronization of the active ingredient increases in an adequate manner the external specific surface of the pulverulent product and already constitutes an approach of this problem. However micronization is only suitable for some pharmaceutical farms, such as dispersions or as a load in soft gelatin capsules. It cannot be a general solution for this problem of resorption while some active ingredients are difficult to micronize since they melt to easily or they have a weak chemical structure.

Adding tensio-active agents may increase the solubility of some active ingredients and thus improve the Kinetics of resorption but this does not systematically allows to obtain more high blood levels. Moreover it is frequently necessary to add significant amounts of tensio active agents (from 25 to 50%) to obtain a significative result. This improvement in the passage through the digestive tract appears to be the result of a decrease in the superficial tension involving an increase in the permeability of the digestive mucosa. However these significant amounts of tensioactive agents are frequently followed with a laxative effect which does not contribute to a good resorption.

Another approach consisted in adding an emulsifying agent and namely a fatty acid ester of a hydrocarbon (sucrose ester) and involves another principle. This ester increases the lipophily of the molecule and makes easier the passage through the intestinal barrier. However this kind of processing only give conclusive results with very lipophilic molecules and needs high concentration of this ester.

More recently, it has been tested in order to improve the resorption of active ingredients, the solubility of which in the biological liquids is poor, to increase the kinetics of dissolution while producing solid dispersions. These solid dispersions—such as those defined by Chiou et al. in J. Pharm. Sci. 60 (1964) 1281-1302—consisting to achieve a system which is a function of the process used in view of the production, may present various structures (see Fort et al. Pharm. Helv. Acta Helv. 62 (1987) 23-27) corresponding to various crystalline states. The vitreous state—whilst it is considered as a solid state—includes a liquid phase which contributes to its structural disorder. This vitreous state is slightly ordered and easy to break. It contributes in a significant manner to an increase in the speed of dissolution, namely for the substances poorly soluble in the aqueous media. However, in spite of the great number of literatures in relation with the production of solid dispersions, more specially those based on Macrogols or Poloxamers, this technique has known only a limited development due to the difficulty to generalize it. In some cases, the dissolution speed is important. In other cases, the dissolution speed is lower and tends to reach an asymptotic value

Therefore, for a same active ingredient and at similar concentrations, it has been observed very significant variations in the speed of solubilization, depending on the nature of the co-solubilizing agent. It has been even observed in some cases , the inability to reach a complete solubilization of the active ingredient, even after a protracted set of time of contact or, at the most, a tendency to recrystallization.

Another way to approach this problem has been the achievement of solid dispersions of a therapeutic agent in a hydrophilic vehicle having an increased solubility in the aqueous medium. This approach consists at first in dissolving the therapeutic active ingredient in a very volatile organic solvent in which a very hydrophilic polymer—such as poly vinyl pyrrolidone—has been added. Thereafter, the solvent is distilled off to dryness in order to produce a co-precipitate of the therapeutic agent and the hydrophilic polymer.

This method has allowed to obtain a clear improvement of the absorption kinetics but it is not suitable for any kind of active ingredient. Moreover, this technique often needs the admixture to the solution, of a tensio active agent which increases the ability to wettening by the digestive media and, optionally, limit the phenomenon of production of crystals which occurs during the storage of solid dispersions. The production of crystalline products contributes to a decrease in the speed of dissolution as a function of time (see Kigudin et al. Chem. Pharm. Bull 9 (1961) 866-872, Duchene D. Pharma 1(11) (1985) 1 064 1073).

From the other side, this technique necessitates the production of co-precipitates in a very hydrophilic lactame such as poly vinyl pyrrolidone having a molecular weight ranging from 10.000 to 5000 and in an oxygenated or chlorinated solvent or mixtures of these solvents (see U.S. Pat. No. 5,776,495).

A more recent approach can be found in the document WO 2005/034920 in the name of Life Science which discloses the technique “Melt dose”. This technique allows a better oral resorption of the molecules having a poor solubility in water. This technique consists in dissolving the molecules in a solvent which facilitates the penetration in the epithelium of the digestive tract and allows the passage in the blood stream.

The solvent disclosed in this document is a vehicle which may be hydrophobic or hydrophilic, which is miscible with water, and which possesses a melting point lower than 250° C. The preferred solvent is a poly ethylene glycol admixed or not with Poloxamer 188.

Polyoxyethylene 32 stearate is not mentioned in this document, and the inert material needed for the production of a powder is not the microcrystalline cellulose but lactose.

This invention carries a much more simple and a much more satisfactory solution to the problem of solubilizing and of intestinal resorption for active ingredients which are poorly or not soluble in water, or for those which cannot be converted into a salt in the gastric juice.

The process according to the invention consists in achieving a dispersion of one or several active ingredients in a fatty acid ester of a poly oxyethylene 32 which can easily melt. This dispersion is sprayed in the hot on a granular excipient in a fluidised bed. The thus formed powdery mixture is divided up in pharmaceutical compositions after optional dilution with an inert, non toxic, pharmaceutically acceptable carrier.

The expression “which can easily melt” means that this ester melts down under 80° C. and, more particularly, between 40 and 50° C.

In a preferred embodiment of this invention, it is used as fatty acid ester of poly oxyethylene 32, a distearate of poly oxyethylene 32, the melting of which occurs in the neighbourhood of 50 to 60° C.

The distearate of poly oxyethylene 32 is a commercially available product. A product of the same family is sold under the Trade Name Kessco® PEG 1540 (Stepan).

According to another preferred embodiment, the granular excipient is an inert material such as cellulose, dextrane, colloidal silica, polymers or copolymers of vinyl pyrrolidone, the poly vinyl pyrrolidone, acrylic polymers such as poly carbophil and the similar products.

A granular preferred material is a microcrystalline cellulose and preferably the pharmaceutical quality sold under the denomination AVICEL PH and particularly the quality sold under the denomination AVICEL PH 105.

The content of active ingredient dispersed in the fatty acid ester of poly oxyethylene 32 may vary in a broad range since these esters are very good solvents. It is thus possible to achieve diluted solutions as well as concentrated solutions.

A preferred concentration in active ingredient ranges from 30 to 50% of active ingredient in the fatty acid ester. Such contents allow to go easily to a solution. A preferred concentration is the one made of 40 to 50% of active ingredient.

Among the active ingredients which it is possible to incorporate in the composition according to the invention, it may be mainly cited:
Anti inflammatories and analgesics
   Salsalate
   Benorylate
   Oxametacine
   Indometacine
   Paracetamol
   Piroxicam
   Tienilic Acid
   Ethenzamide
   Tramadol
Immunosuppressives
   Cyclosporine
   Tacrolimus
Antihistaminics
   Terfenadine
   Brompheniramine
   Chlorpheniramine
antifungal and anti trichomonas agents
   Metronidazole
   Ornidazole
   Dapsone
   Itraconazole
   Terbinafine
antiviral agents
   Cytarabine
   Gancyclovir
   Acyclovir
antipsychotic agents
   Sulpiride
   Sultopride
   Amisulpride
hormones
   Estradiol and its esters (17β-valerate)
   Estrone
   Estriol
   Progesterone and its derivatives
cardio vascular agents and vasodilatating agents
   Dobutamine
   Diltiazem
   Nifedipine and its analogs (Nitrendipine, Nisoldipine . . . )
antiulcerous agents
   Pirenzépine
   Ranitidine
   Oméprazole
   Lansoprazole
antibacterial products
   Erythromycine
   Flumequine
   Oxytetracycline
   Piperacilline
   Cefuroxime
   Amphotéricine
anti arythmic agents
   Propafenone
   Amiodarone
   Cordarone
   Flecainide
   Gallopamil,
   Verapamil,
   Dipyridamole,
   Diisopyramide
uricosuric agents
   Benzbromarone
   Probenécid
   Sulfinpyrazone
   Allopurinol
antimigrainous agents
   Flunarizine
   Derivatives of Ergotamine
   Sumatriptan
antidepressive agents
   Fluvoxamine
   Fluanisone
   Fluoxétine
   Paroxétine
anti hormonal agents
   Flutamide
bronchodilatating agents
   Tulobutérol
   Talinolol
   Prenaltérol
anxiolytics
   Thiothixène
   Trazodone
   Doxepine
   Carbamazépine
coronarodilatating drugs
   Ethaverine
   Pentoxyphylline
   Eburnamonine
Diuretic drugs
   Furosemide
   Triamterène
   Torasemide
   Hydrochlorothiazide
antispasmodic agents
   Flavoxate
   Trimébutine
   Phlorogucinol
agents inhibiting the excretion of calcium
   Clodronate
   Pamidronate
   Alendronate
anticlotting agents
   Pindione
   Tromexan
antalgics
   Fentanyl,
   Dextropropoxyphène,
   Sufentanyl.
opiate drugs
   Nalbuphine,
   Naltrexone,
   Dihydrocodéïnone,
   Buprenorphine,
   Méthadone.

This invention finds a very specific use for the realization of pharmaceutical formulations the bio availability of which is greatly improved and the active ingredient of which is an anti lipaemic or a hypocholesterolemizing agent.

It may be more precisely cited the compositions based on clofibric acid or fenofibric acid such as for example clofibrate, fenofibrate, gemfibrozil, bezafibrate, ciprofibrate, pirifibrate or simfibrate.

It may also be cited the HMG.Co A reductase inhibitors (statines) such as Atorvastatine, Cerivastatine, Fluvastatine, Pravastatine and its Sodium salts, or Simvastatine and even triptans such as Sumatriptan or Frovatriptan.

In the specific case of clofibric acid derivatives, the interest of a solvent such as a fatty acid ester of poly oxyethylene 32 lies in the fact that the compound is not likely to promote or produce a transesterification or even to increase the toxicity of the active ingredient.

Another feature of this invention is to be in a position to achieve bioavailable formulations of hormonal products which are poorly or not resorbable in the digestive tract such as progesterone, androsterone, chlormadinone acetate, or melengestrol. Usually one utilizes the alkyl derivatives in position 17α—or in position 6α, in order to obtain compounds which are active through the digestive way (Cyproterone, Demegestone, Promegestone, Nor ethynodiol acetate, Ethynyl estradiol). This substitution shows the inconvenience to induce noxious side effects (androgenic or anti-androgenic effects, and all the most hepatoxic effects) which have more precisely to be avoided. This is the reason why natural progesterone or its derivatives (dihydroprogesterone, 17α-hydroxyprogesterone) are used. The latter ones being active through digestive way and weakly toxic, but however less toxic than progesterone itself.

The dispersions according to the invention allow the achievement of pharmaceutical compositions containing in addition to the active ingredient dispersed on the inert carrier, one or more inert, non toxic, pharmaceutically-acceptable excipients.

It may thus be possible to add to the obtained preparations after incorporation into an inert material followed by a drying on fluidized bed, diluting agents, loading agents, aromatizing agents, colouring matters, desagregation agents, gelifying agents and/or film-forming agents. The content into active ingredient is calculated in such a manner that the final pharmaceutical composition contains an efficient and non toxic teneur in active ingredient. The amount of carrier is calculated in order that the concentration into active moiety be of the order of 50% i.e. it does not pass 50%, being preferably comprised between 20 and 40%.

A specific example resides in the preparation of pharmaceutical compositions based on adsorbates of fenofibrate in microcrystalline cellulose. In these compositions the content into active ingredient ranges from 50 to 150 mg per unit dosage and preferably 60 mg, 90 mg or 130 mg of fenofibrate.

The content into microcrystalline cellulose ranges from 40 to 60 mg per unit dosage.

The content into polyoxyethylene 32-distearate will also be from 5 to 100 mg per unit dosage and the content into carrier will also be comprised between 5 and 100 mg.

Some compositions based on fenofibrate, based on progesterone, or based on amiodarone are provided hereafter as examples. They do not limit in any manner the scope of this invention.

### EXAMPLE I

| | |
|---|---|
| Fenofibrate | 25 g |
| Polyoxyethylene 32 distearate | 26 g |
| Microcristalline cellulose | 44 g |
| Magnesium stearate | 0.5 g |

This so-produced mixture is divided into 1000 soft gelatine capsules containing 25 mg fenofibrate per unit dosage.

### EXAMPLE II

| | |
|---|---|
| Progesterone | 30 g |
| Polyoxyethylene 32 distearate | 25 g |
| Microcrystalline cellulose | 55 g |
| Maltodextrine | 12 g |
| Calcium carbonate | 5 g |
| Talc | 5 g |

For 1000 soft gelatine capsules each containing 30 mg progesterone.

### EXAMPLE III

Amiodarone

Polyoxyethylene 32 distearate

Polyvinylpyrrolidone

Colloïdal silica

Rice starch

For 1000 tablets each containing 150 mg Amiodarone

This invention has also as the subject matter production of sublingual formulation and of buccal tablets. They are intended to be placed under the tongue for the sublingual tablets or sticked to the palate for the buccal tablets. These kinds of tablets are realized under the process of this invention and show a still increased bio availability.

These suglingual or buccal tablets are produced by means of the process according to the invention but the powdery granular product is thereafter converted into tablets by adding a binding agent, a compression agent and a gel forming agent.

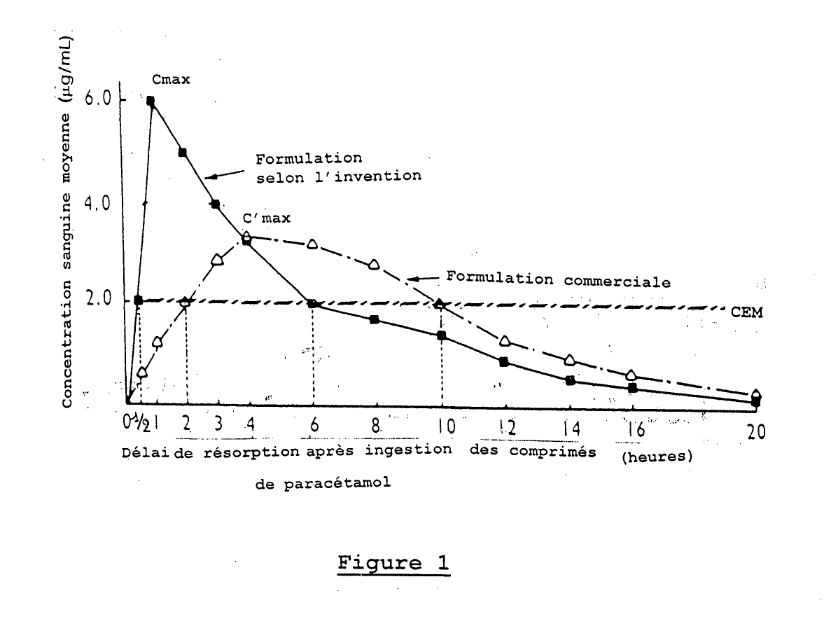

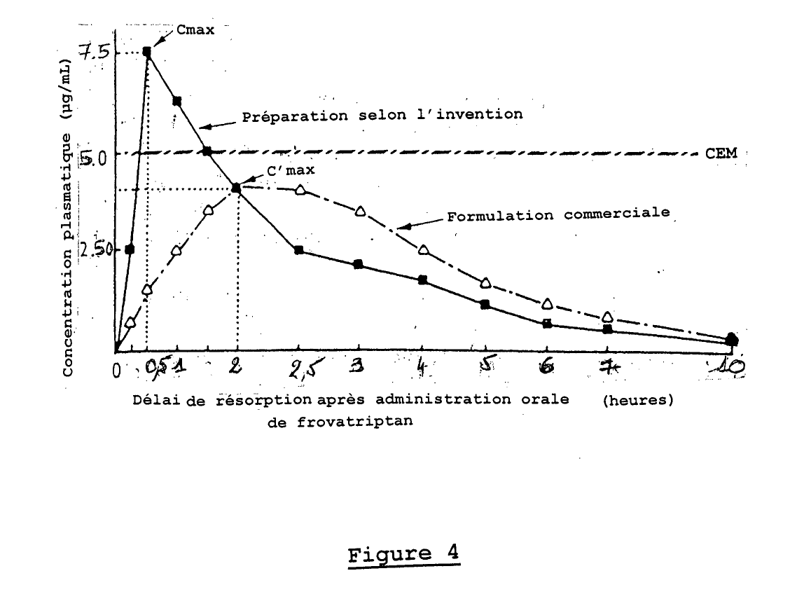

FIGS. 1 to 4 show the results of the pharmacoKinetic studies performed with the compositions in accordance with the invention for many active ingredient. The concentrations of minimal efficiency (CEM) are indicated for each compound. In the following examples the terms formulation or preparation are equivalent to the terminology “compositions”.

FIG. 1 is a graph showing the mean blood concentration (in μg/ml) as a function of the time of resorption after ingestion of tablets of paracetamol (expressed in hours). For the formulation in accordance with the invention the results are obtained with a composition based on 350 mg paracetamol produced in accordance with the process of this invention.

These results have to be compared to those obtained with commercial tablets containing the same doses of paracetamol.

It appears from this figure that in the case of the formulation according to the invention the concentration peak is reached only 1 hour after ingestion of paracetamol tablet and that the maximal concentration (Cₘₐₓ) is then lower than 4.0 μg/ml.

From another side the CFM of paracetamol is reached in only ½ for the composition based on paracetamol according to the invention while this concentration is only reached after 2 hours with the marketed composition.

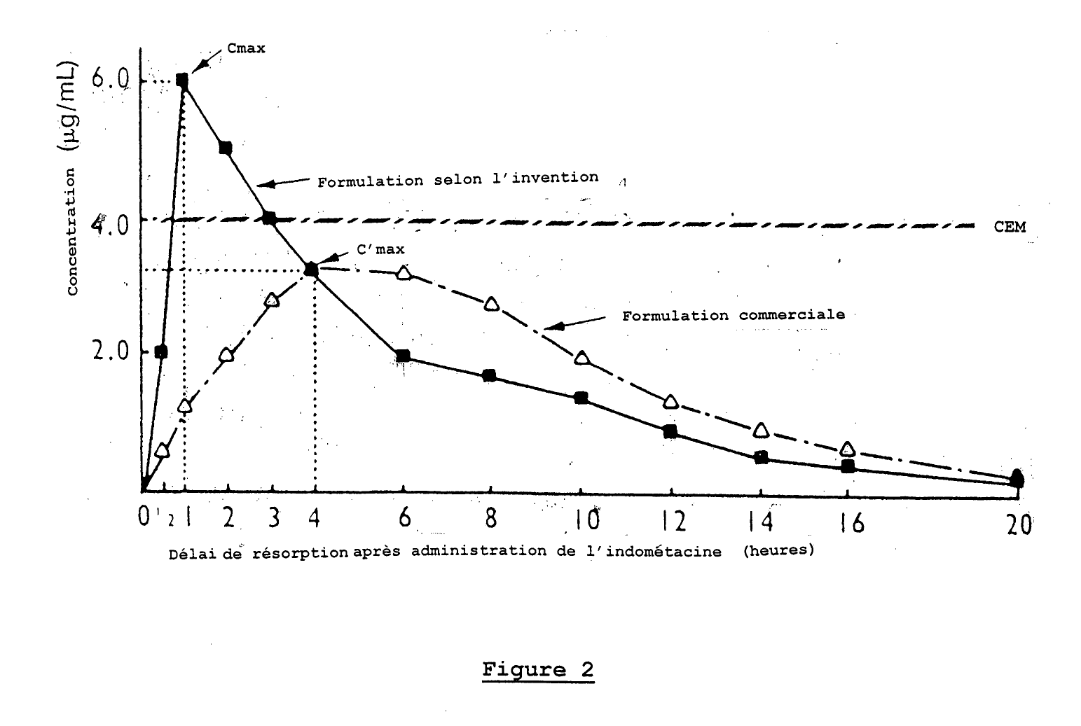

FIG. 2 is a graph picturing the blood concentration (in μg/ml) as a function of the time of resorption after administration of indometacine (expressed in hours). This picture illustrates the obtained results with a formulation produced in accordance with the process of this invention, and the active ingredient of which is indometacine, compared to the results obtained with a formulation of indometacine of the market.

Form the curves shown here, the maximal peak concentration appears in a short time owing to the fact it appears merely one hour after the administration of indometacine. Moreover the maximal concentration (Cₘₐₓ) observed at the time of this peak, is 6 μg/ml for the formulation in accordance with the invention. For the marketed composition it is necessary to wait for 4 hours after the administration to observe the blood concentration peak and the maximal concentration (Cₘₐₓ) of the marketed composition is then lower than 4 μg/ml. Moreover the indometacine CEM is reached 3 hours after administration of the composition in accordance with the invention while the concentration is never reached with the marketed composition.

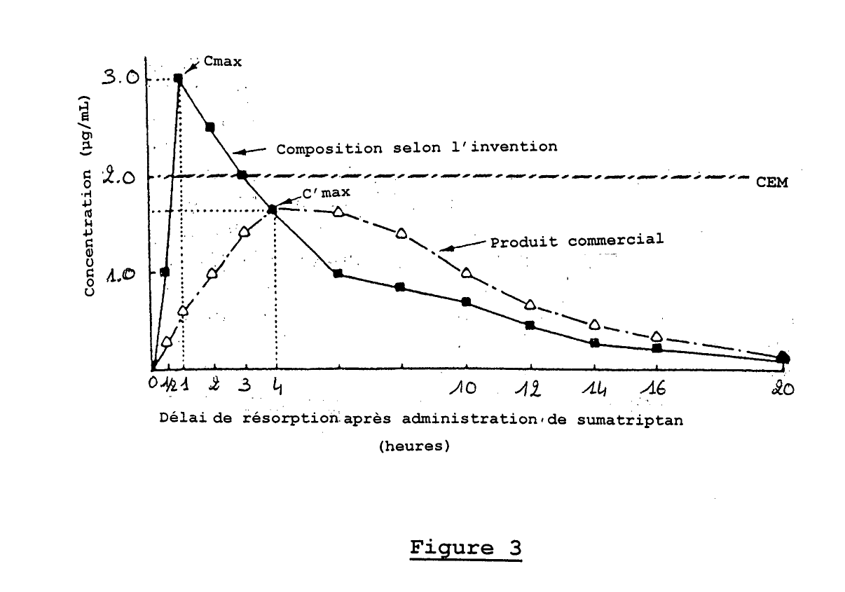

FIG. 3 is a graph showing the blood concentration (expressed in μg/ml) as a function of the time for resorption after administration of sumatriptan (expressed in hours). The curves illustrate the obtained results with a composition in accordance with the invention, the active ingredient of which is sumatriptan compared with those obtained with the marketed product. One can see in this graph that the blood concentration peak is only reached one hour after the administration of the composition in accordance with the invention and the maximum concentration (Cₘₐₓ) is then of 3 μg/ml.

In the case of the marketed product the blood peak appears only 4 hours after administration and the maximal concentration (C′ₘₐₓ) in lower than 2 μg/ml for this marketed product. On the other side, the CEM for sumatriptan is reached 3 hours after administration of the composition in accordance with the invention while with the marketed product, this CEM is never reached.

1. A method for producing dry hydrodispersible pharmaceutical formulations wherein one or several active ingredients are dispersed in a fatty acid ester of polyoxyethylene 32 which melts below 80° C., then the said dispersion is sprayed in the hot on a granular carrier in a fluidised bed to be converted into pharmaceutical compositions in a per se known manner.

2. A method according to claim 1 wherein the fatty acid ester of polyoxyethylene 32 is the distearate of polyoxyethylene 32.

3. A method according to claim 1 wherein the granular carrier is an inert material.

4. A method according to claim 1 wherein the granular carrier is the microcrystalline cellulose.

5. A method according to claim 1 wherein the fatty acid ester of polyoxyethylene 32 is a distearate of polyoxyethylene 32 the melting point of which lies between 50 to 60° C.

6. A method according to claim 1 wherein the concentration into active ingredient in the fatty acid ester of polyoxyethylene 32 ranges from 30 to 50%.

7. A method according to claim 1 wherein the concentration into granular material per unit intake of the amount of carrier is determined in order the concentration into active ingredient is in the order of 50%.

8. A method according to claim 1 wherein in view of the production of soft gelatine capsules of fenofibrate at 25 mg of active ingredient, the amount of microcrystalline cellulose is of the order of 0.044 g per soft gelatine capsule.

9. A method according to claim 1 wherein in view of the production of soft gelatine capsules of progesterone at 30 mg, the amount of micro crystalline cellulose is in the order of 0.055 g per soft gelatine capsule.

10. A method according to claim 1 wherein the active ingredient or the mixture of active ingredient(s) is (are) admixed with one or several inert, non toxic pharmaceutically acceptable carrier(s), in view of the production of pharmaceutical composition.

11. A method according to 1 wherein the active ingredient is fenofibrate dissolved in polyoxyethylene 32 distearate, then dispersed on a carrier based on micro crystalline cellulose.

12. A method according to claim 1 wherein the active ingredient is progesterone dissolved in polyoxyethylene 32 distearate then dispersed on a carrier based on microcrystalline cellulose.

13. A method according to claim 1 wherein the active ingredient is a statine dissolved in polyoxyethylene 32 distearate then dispersed in a carrier based on microcrystalline cellulose.

14. Pharmaceutical compositions intended for oral administration containing as active ingredient the hydrodispersible composition of claim 1 admixed with one or several carriers suitable for digestive administration.

15. Pharmaceutical compositions according to claim 14 wherein the hydrodispersible composition is a solution of fenofibrate into polyoxyethylene 32 distearate.

16. Pharmaceutical composition according to claim 14 wherein the hydrodispersible composition is a solution of progesterone into polyoxyethylene 32 distearate.

17. Pharmaceutical composition according to claim 14, wherein the hydrodispersible composition is a solution of Amiodarone into polyoxyethylene 32 distearate.

18. Pharmaceutical compositions according to claim 14 wherein the active ingredient is buprenorphine or one of its salts dissolved into polyoxyethylene 32 distearate.

19. A method according to claim 1 wherein the pharmaceutical composition is in the form of sublingual tablets or buccal tablets.

20. (canceled)
